(19) United States
(12) Patent Application Publication
(10) Pub. No.: US 2015/0165400 A1
(43) Pub. Date: Jun. 18, 2015

(54) CRYSTALLISATION PROCESS

(71) Applicant: University of Bradford, Yorkshire (GB)

(72) Inventors: Chaitrali Kulkarni, Yorkshire (GB); Ravindra Dhumal, Yorkshire (GB); Adrian Kelly, Yorkshire (GB); Tim Gough, Yorkshire (GB); Anant Paradkar, Yorkshire (GB)

(21) Appl. No.: 14/400,946

(22) PCT Filed: May 15, 2013
(86) PCT No.: PCT/GB2013/051248

§ 371 Date: Nov. 13, 2014

(30) Foreign Application Priority Data
May 15, 2012 (GB) 1208489.3

(51) Int. Cl. B01F 7/08; C07C 311/58; C07D 223/26; C07D 207/27; C07D 493/18; C07C 303/42

(52) U.S. Cl. CPC B01F 7/081; C07D 493/18; C07C 303/42; C07D 223/26; C07D 207/27; C07C 311/58; B01F 2215/0032

(57) ABSTRACT

The present invention relates to a method useful for forming products which are useful in a pharmaceutical context, and products formed by such a method. The invention relates particularly, but not exclusively, to methods of forming a metastable polymorph using screw extrusion, whereby temperature and shear induce transformational changes, and products obtained or obtainable via such methods.

The present invention relates to a method for forming products which are useful in a pharmaceutical context, and products formed by such a method. The invention is more particularly concerned with polymorphic forms of active pharmaceutical ingredients, commonly known as APIs. The invention relates to methods of forming a metastable polymorph using screw extrusion, whereby temperature and shear induce transformational changes in the crystal structure.

A metastable state for such a polymorphic crystal means that the crystal structure i.e. the particular polymorphic form, is in a state of apparent equilibrium although it is capable of changing to a more stable state. It can be exceedingly difficult to produce metastable polymorphic forms when more stable crystalline polymorphic forms are available to the molecule, and even if obtained, the resulting metastable polymorphic form may be impure and is often quite short-lived.

The invention provides metastable polymorphic forms which are stable in the sense that they do not undergo any significant transformation to a more thermodynamically stable polymorphic form of the same material over an extended period of time such as days, months or even years. The invention also provides for products obtained or obtainable via such methods.

### BACKGROUND

Crystal engineering has been investigated recently as a means of tailoring the physicochemical properties of active agents. Its application to pharmaceuticals provides a new path for the systematic discovery of a wider range of new crystal forms (solvates, salts, molecular salts, co-crystals and polymorphs) with properties differing when compared to pure, amorphous active pharmaceutical ingredients (APIs). Crystal engineering provides an interesting potential alternative approach available for the enhancement of drug solubility, dissolution, melting points, moisture uptake, physical and chemical stability and bioavailability.

The ability of a solid compound to exist in more than one crystal form is known as polymorphism. A polymorph is a solid crystalline phase of a compound resulting from the possibility of different crystalline arrangements and packing of molecules in a crystal lattice. Solid state properties of drugs have drawn a lot of attention in recent years and have made an exciting platform for many researchers. It has been proved from recent studies that 80 to 90% of organic molecules exist in polymorphic forms (Stahly G., Cryst. Growth Des. 7 (2007) 1007-1026).

Most marketed drugs are in a crystalline state. Each crystal packing with different molecular arrangement in a unit cell possesses a unique set of physical properties. This includes melting point, solubility, density, flowability, vapour pressure, surface properties, hardness, stability, dissolution and bioavailability. These distinct properties impact on the processing and formulation of drugs that have received attention from pharmaceutical industry. The important factor in solid state research is to identify all significant polymorphs, and characterise and select the most appropriate polymorphs for further pharmaceutical development. Nowadays, research on polymorphism and its material properties is an important stage of drug development.

Several examples are present in the pharmaceutical industry where different crystal forms of a particular compound greatly affect quality and stability of the product. For example paracetamol, the well-known antipyretic drug exists in two polymorphic forms; orthorhombic and monoclinic. The commercially available monoclinic form of paracetamol has poor compressibility and many pharmaceutical companies are interested in the directly compressible orthorhombic form of paracetamol. Another example is theophylline, which exhibits four polymorphic forms and all forms have diversity in their packing properties. These examples serve to show that it is important to prepare the required polymorphic form, as another form may not have the desired properties.

Once a desired polymorphoric form has been identified and prepared it is essential to maintain the API in the desired polymorphoric form. Therefore, polymorph stability over a period of time is a main concern, especially as uncontrolled transformation of one polymorph to another is extremely undesirable. Because energy differences between polymorphs are relatively low, such inter-conversion from one polymorph to another more stable form is inherent and is catalysed by the presence of impurities including impurities in the form of other polymorphic forms which may also be present. Therefore, typically, in many commercial dosage forms, a more thermodynamically stable polymorphic form is preferred. However, some thermodynamically stable forms experience issues with solubility, bioavailability, manufacturing processes, and chemical stability.

Traditional methods to control growth of stable crystal polymorphic forms include the addition of additives, seeding crystallisation, contact line crystallisation, and mechanical stress. However, such methods are difficult to control specifically to avoid the formation of more than one polymorph and are generally limited to small volumes. The preferred industrial crystallisation route is from solution, because the crystals tend to have higher purity and the process can be easily scaled up from laboratory to much larger quantities. However, it is known that although a less stable polymorph may nucleate first in solution due to a higher nucleation rate, it is energetically favourable for it to convert to a more stable polymorph over time. Therefore, in many cases, it is difficult to isolate a metastable form before it undergoes a solvent mediated transformation to the more stable form.

Boldyreve et al., (Boldyreve E V, T P Shakhtshneider, H Sowa and H Uchtmann, Journal of thermal analysis and calorimetry 68 (2002) 437-452) demonstrated pressure induced transformation of paracetamol. The study was carried out in a diamond anvil cell. There was no change in a single monoclinic form crystal till pressure was increased up to about 4.5 GPa. Sometimes the transformation was observed when the pressure was slowly decreased after initial increase. Overall this transformation is poorly reproducible and depended strongly on the sample and on the procedure of increasing/decreasing pressure.

Haisa et al., (Haisa, S. Kashino, R. Kawai and H. Maeda, ActaCryst. B32 (1976) 1283-1285) have obtained metastable orthorhombic paracetamol polymorph by slow evaporation in ethanol solution but this method is not reproducible (Haisa et al., 1976). Another study indicated polymorphic transformation of pentaerythritol under pressure. The powder sample of pentaerythritol underwent polymorphic transformation at 500 MPa, whereas its single crystal required pressures higher than 1.5 GPa (Katrusiak A, Acta Cryst. B5 (1995) 873).

Francesca et al., (Francesca P A, Fabbiani, David R Allan, William I F David, Stephen A Moggach, Simon Parson, Colin R Pulham, Cryst. Eng. Conun. 6 (2004) 504-511) have reported a high pressure recrystallisation method to generate novel polymorph and phenanthrene from dichloromethane at pressure 1.1 GPa and the crystallisation of a novel dehydrate of paracetamol from water at 1.1 GPa pressure.

Otsuka et al., (Makoto Otsuka, Takahiro Matsumoto, Shigesada Higuchi, Kuniko Otsuka, Nobuyoshi Kaneniwa, J. Pharm. Sci. 84 (1995) 614-618) have reported polymorphic transformation of chlorpropamide form A to form C during tableting. However, this conversion is partial and depends upon pressure distribution.

Similarly, Brittain (H. G. Brittain. J. Pharm. Sci., 91 (2002) 1573-1580) demonstrated a polymorphic transformation in the crystal structure of caffeine during a mechano-chemical process which was directly proportional to the degree of applied pressure and generated temperature. However, complete conversion was not achieved and the process was not reproducible.

Haiyan et al., (Haiyan Qu, Katherine Bisgaard Christensen, Xavier C. Frette, Fang Tian, Jukka Rantanen, Lars Porskjaer Christenesen, Chem. Eng. Technol. 5 (2010) 791-796) carried out crystallisation of artemisinin with the use of an antisolvent, and evaporative and cooling crystallisation methods. It was observed that formation of polymorph depended on the solvent and rate of generation of supersaturation. In the antisolvent technique, water was added as an antisolvent in the solution of artemisisnin in acetonitrile and acetone. Antisolvent crystallisation from acetonitrile always yielded stable orthorhombic form irrespective of rate of addition of antisolvent whereas triclinic form was generated first during fast antisolvent addition to acetone which underwent solvent mediated transformation to the stable orthorhombic form. During fast evaporative crystallisation from ethyl acetate solution, the triclinic form of artemisinin was generated whereas evaporation from other solvents such as dichloromethane, acetone, acetonitrile, ethanol, methanol, hexane, 1 butanol, 1-propanol, 2 propanol and chloroform resulted in formation of the orthorhombic form.

Louer et al., (Louer D, Louer M, Acta. Cryst. B51 (1995) 182-187) generated metastable piracetam polymorph at room temperature. The form I of piracetam was formed by heating form III to 410K at ambient pressure for 30 minutes in a glass capillary followed by quenching to room temperature. This technique was suitable for laboratory scale.

Rene et al., (Rene Ceolin, SiroToscani, Marie-France Gardette, Viatcheslav N. Agafonov, Aleksander V. Dzyabchenko, Bernard Bachet, J. Pharm. Sci. 86 (1997) 1062-1065) reported the first crystallographic information on triclinic carbamazepine crystals. A monoclinic carbamazepine sample was placed at one end of silica tube and the sample was allowed to sublime by placing the sample in the middle of a horizontal furnace; the other end of the silica tube was laid out of the furnace at room temperature. The furnace was heated at 2° C. min⁻¹ up to 150° C. and phase transformation was observed after two weeks. It has been noted that polymorphic conversion of monoclinic to triclinic is strongly dependant on kinetic factors. No transformation occurred at heating rate 10° C. min⁻¹. The same authors found similar results when performed in a DSC apparatus.

Gaisford et al., (Gaisford S, Buanz A, Jethwa, J, Pharmaceutical and biomedical analysis, 53, (2010) 366-370) prepared and characterised the most unstable polymorph of paracetamol. Paracetamol polymorph III was prepared from glass by heating form I to 180° C. and holding isothermally for about 5 minutes. The experiment was conducted in the presence of a growth modifier hydroxypropylmethylcellulose which act as a stabiliser and it was observed that increasing amount HPMC resulted in significant increase in the polymorphic transformation. The stabilisation of metastable form by adding polymer may be attributed to a specific interaction between the drug and the polymer.

Yuen et al., (Yuen Kan-Hay, Kit-Iam Chan, Hiroaki Takayanagi, Sunil Janadasa and Kok-KhiangPeh, Phytochemistry 46 (1997) 1209-1214) described polymorphism of artemisinin from Artemisia annua. Artemisinin was recrystallised several times from cyclohexane and ethanol to produce triclinic form and orthorhombic form respectively. The yield of both forms were very low, for triclinic crystals 0.39% whereas for orthorhombic crystals 0.24%. The triclinic crystals showed four times faster dissolution rate in comparison with orthorhombic crystals.

Grant et al., (Grant J W, Young Victor, Chatterjee Koustuv, Chong-HuiGu, J. Crystal growth 235 (2002) 471-481) have reported stabilisation of a metastable polymorph of sulfamerazine by structurally related additives. They have studied solvent mediated transformation (I→II) of sulfamerazine in acetonitrile solvent. The rate of conversion was controlled by adding N4-acetylsulfamerazine, sulfadiazine or sulfamethazine. The concerns have been raised because success rate is low as they do not consider kinetic factors affecting crystallisation.

Crystallisation of metastable polymorph of paracetamol has been noted to occur around the edges of an evaporating aqueous solution by Capes et al. (Capes S J, Cameron Ruth E, Crystal growth and design 7 (2007) 108-122). The paracetamol form I and water were placed in a sample dish and kept in a custom-made aluminium insert in a block heater. The temperature range was 40° to 80° C. and the solution was heated for about 4 minutes. At the end of heating time dish was removed onto a cool metal block and cooled rapidly to room temperature. The solution was allowed to freely evaporate at room temperature and the experiment was repeated ten times. Interestingly the obtained form was stable for several months but the method suffers from scale-up difficulties.

Seeding crystallisation is common technique to induce crystallisation and achieve metastable polymorph. A general method to achieve metastable form is by quenching the pure substance in liquid form. However, this is not a deliberate method because the interval of the metastable zone should be known to harvest seeds of metastable polymorph (Beckmann et al., Crystal growth and design 3 (2003) 959-965). Another issue is with complete drying because residual solvents may facilitate a conversion to the most stable polymorph.

Shan et al. (Shan-Yang Lin, Wen-Ting Cheng, J. Pharm. Sci. 2 (2007) 211-219) studied the effect of environmental humidity and moisture on the polymorphic transformation of famotidine in the grinding process. The relationship between molecule and solvents as well as guest and host molecule determines the desired polymorph. They have reported that the water contained in famotidine form B promotes the rate of polymorphic transformation. However, this method is not reproducible and depends upon environmental conditions.

Mitchell et al., (C. A. Mitchell, L. Yu, M. D. Ward, J. Am. Chem. Soc. 123 (2001) 10830) have explained selective nucleation and discovery of an organic metastable polymorph of 5-methyl-2-[(2-nitrophenyl)amino]-3-thiophenecarbonitrile through Epitaxy with single crystal substrate. 5-methyl-2-[(2-nitrophenyl)amino]-3-thiophenecarbonitrile forms six different polymorphs from solution but selective orientation was different on single crystal pimelic acid substrate. When freshly cleaved various faces of pimelic acid such as (101)PA, (111)PA and (010)PA were exposed, different observations were found. The growth of YN metastable polymorph was found on the (101)PA face, no orientation was observed on the (111)PA face whereas the (010)PA surface was less selective, and promoted growth of several polymorphs. This data indicated that polymorph growth is highly sensitive on the surface of the substrate.

Lee et al., (E. H. Lee.; S. R. Byrn, and M. T. Carvaja, Pharm Res. 23 (2006) 10) have reported that multiple crystal forms of a compound can be formed on patterned self-assembled monolayer substrate in a solvent system. They used mefenamic acid and sulfathiazole as a model drug. Based on different wetting properties an array of small solution droplets at the nanoscale was formed on the substrate. Different droplet dimension were deposited on the substrate and as solvent evaporated from droplets, crystals were formed with controlled volume. The produced crystals were characterised by Raman spectroscopy.

US patent application number 2005/0256300 discloses the application of a strong static electric field to obtain desired polymorphs of organic molecules from saturated solutions.

U.S. Pat. No. 7,122,642 discloses a method of producing unexpected polymorphs of organic molecules from supersaturated solutions using non photo-chemical laser induced nucleation.

US patent application US 2011/0021631 discloses a method to avoid polymorphic transformation in atrovastatin and its salts during processing and in formulation. It was demonstrated that polymorphic transformation of pravastatin sodium in the presence of a wet phase can be prevented using microcrystalline sodium.

US patent application number US2007/0224260 discloses the method of preparation of mini-tablets containing drugs which can be formed at lower pressure thereby avoiding polymorphic transformation during processing.

Patent application US 2011/0177136 reported the use of twin screw extrusion technology for generation of co-crystals. It has also been reported in some publications such as Medina et al., for formation of co-crystals of caffeine and AMG 217 (Medina C, Daurio D, Nagapudi K, Alvarez-Nunez F.-, J Pharm Sci. 2010;99:1693-6.); Dhumal et. al (Dhumal R, Kelly A, York P, Coates P. and Paradkar A; Pharm Res (2010) 27:2725-2733); and in Kelly et. al (Kelly A, Gough T, Dhumal R, Halsey S and Paradkar A, International Journal of Pharmaceutics 426 (2012) 15-20). The process involves the application of shear to a mixture of API and conformer in a molar ratio maintained at suitable temperature which is near to the eutectic point or melting point of the lower melting component. The co-crystals formed have a completely different composition compared to the polymorphs. Co-crystals are multi component systems whereas polymorphs are single component systems. One of the basic requirements for formation of a co-crystal is formation of a non-covalent bond such as hydrogen bond, or a hybridised [sp] bond between two components of the system. There is no such requirement in the formation of polymorphs because they are single substances which are conformationally rigid molecules that are packed into different three-dimensional structures in the absence of a bond.

There is thus a need for less thermodynamically stable (metastable) polymorphic forms of certain APIs (relative to more thermodynamically stable forms of the same API) since these metastable forms may have improved properties relative to other, more thermodynamically stable forms. The invention therefore aims to provide metastable polymorphic forms of APIs that have one or more improved properties relative to a thermodynamically more stable polymorph of the same API. Such improved properties include improvements in: solubility, bioavailability, flowability, compressability, colour, ease of formulation, simplicity and or convenience of the manufacturing processes, and chemical stability.

A further aim is to provide metastable polymorphic forms which are stable on storage, over an extended period of time under ambient conditions or conditions of elevated temperature and or humidity in more extreme climates. It is another aim to provide metastable polymorphic forms which are substantially pure in the sense of being substantially free of other polymorphic forms. Ideally, the metastable polymorph should also be substantially free of other impurities.

There is also a need for process for reliably and conveniently preparing metastable polymorphic forms. It is an aim to provide a process which does not require the use of an anti-solvent. A further aim is to provide a process which can be carried out without the need for any solvent i.e. a solvent-free process for effecting formation of the metastable polymorphic form. Ideally, the process will not require any seeding to be carried out. A further aim of the invention is to provide an economical process for preparing metastable polymorphic forms.

The present invention satisfies some or all of the above aims.

### BRIEF SUMMARY OF THE DISCLOSURE

According to a first aspect, the present invention provides a solvent-free method of forming a metastable polymorph of a pharmaceutical active ingredient, the method comprising the steps:

(a) placing an amorphous or crystalline compound into an extruder,

(b) providing a source of heat to the extruder to heat the compound to a temperature in the range from 8° C. below the melting point of the compound to 20° C. below the melting point of the compound, and

(c) extruding the compound from the extruder.

If necessary, the melting point of the compound can be determined separately before the extrusion process is conducted to determine an appropriate extrusion temperature range.

The compound is normally an API though the process can be applied to any compound capable of existing in more than one polymorphic form. The API may be an organic compound or an inorganic compound. More usually, the API will be an organic compound.

The temperature to which the source of heat is operative to heat the compound to i.e. a temperature in the range of from 8° C. to 20° C. below the melting point of the compound may be referred to herein as the conversion temperature. For practical purposes, the compound exiting the extruder will be at the same temperature and the extrusion temperature is thus effectively the same as the conversion temperature.

In a preferred embodiment, the source of heat is operative to heat the compound to a temperature in the range of from 10° C. below the melting point of the compound to 15° C. below the melting point of the compound. The initial heating rate from ambient temperature to the desired conversion temperature is controlled to avoid overheating and to avoid any unwanted reversion of the metastable form to an unwanted thermodynamically more stable form. The rate of heating is in the range of from 10° C. per minute to 200° C. per minute.

The extruded material may be held at the conversion temperature immediately after extrusion for a period of time or it may be allowed to cool to room temperature naturally. This period of time may be from 30 seconds to 10 minutes, and more typically will, when used, be 30 seconds to 2 minutes. In some embodiments, cooling may be applied to accelerate cooling beyond the natural rate of cooling. The applied cooling may take place either directly after extrusion or after extrusion followed by maintenance at the conversion temperature. The cooling rate may vary from 50° C. per minute to 200° C. per minute.

Where necessary and appropriate, the extrudate may be returned to the input side of the extruder (or different extruder) and the extrusion process repeated one or more times as required. The or each subsequent extrusion process may be carried out under the same or different conditions from the initial extrusion.

The process can be carried out batchwise or as a continuous process. In another preferred embodiment, the method of the present invention is a continuous method. This means that the method is not a conventional batch process and the compound is extruded continuously and fresh starting material is supplied to the extruder as it is consumed. However, continuous does not mean the method is run without stopping as it may be necessary on occasion to stop the process for a variety of reasons. The ability to perform a synthetic method in a continuous process is a significant advantage compared with conventional batch methods. Advantages over a batch process include improved efficiency, simpler scale-up, consistent product characteristics, the avoidance of lead-times, and reduced need for cleaning.

In a preferred embodiment, an extruder is a twin screw extruder.

The compound resides within the extruder for a particular residence time during which it is heated and then subjected to shear due to the action of the screw or screws in the extruder. In an embodiment, this residence time is between 30 seconds to 15 minutes, more preferably it is between about 5 and 15 minutes, and more usually is from 8 to 12 minutes. In other words, the time from material being introduced into the hopper of the extruder to the point at which it is extruded is usually between 5 and 15 minutes. This applies to both continuous and batch processes.

In a preferred embodiment the substance is an active pharmaceutical ingredient (API), more preferably an organic compound. The process is particularly suited to low molecular weight organic compounds with a molecular weight between 100 and 600. As discussed above there is a particular need for improved methods for producing metastable polymorphs in the pharmaceutical field. Existing technologies in this field suffer from disadvantages including being labour intensive, slow, inconsistent and/or unreliable, not amenable to scale-up or a combination of these problems.

According to a second aspect, the present invention provides a substantially pure metastable polymorphic form of a compound having at least one thermodynamically more stable polymorphic form.

The resulting metastable polymorphic form of the compound is substantially free from impurities. Impurities may include some or all of: other polymorphic forms of the same compound, solvent, and chemical impurities i.e. other chemical compounds or enantiomers/diasteroisomers of the compound. The term “substantially pure” means that there is less than 2%, and preferably less than 1% in total of these impurities. More preferably this is less than 0.5%, 0.1% or even 0.05% in total. Sometimes, all of the impurity present may be accounted by only one or two of the above impurities. In a particularly preferred embodiment, the metastable polymorphic form of the compound is substantially free from solvent, meaning that it contains at most less than 0.5% residual solvent. In another preferred embodiment, there is less than 5%, more preferably less than 2%, and even more preferably less than 1% of any other polymorphic form of the compound present. Ideally there will be less than 0.5%, 0.1% or even 0.05% in total of other polymorphic forms present.

The resulting metastable polymorphic form of the compound is stable relative to conversion to a thermodynamically more stable polymorphic form of the same compound for an extended period of time. In practice, the metastable polymorph is stable for a period of at least 30 days, and more preferably at least 6 months. The more successful polymorphic forms of the invention are stable for at least 12, 18, or 24 months.

According to a third aspect, the present invention provides an apparatus suitable for polymorphic transformation of a substance. This apparatus is normally an extrusion device which includes a source of heat and a high shear mixing means. Usually, high shear mixing is effected using a screw or screws or paddles. It is important in the apparatus of the invention that temperature at which the initial compound is held within the extruder can be precisely controlled. Similarly it is important that the residence time in the apparatus can be accurately controlled and this is governed in part by the screw speed of the extruder screw. The rate of shear is also controlled by the screw speed during extrusion as well as by the screw or paddle design.

As used herein, extrusion can be used to mean a process of forming a product by forcing a material through an orifice or die. This process is normally carried out in a continuous manner by the action of an Archimedean screw rotating in a heated barrel in the case of hot extrusion. For polymers, melting is achieved by the dual action of conductive heating above the polymer melting point through the barrel walls and viscous shearing of the polymer.

The simplest and most widely used form of extruder is that employing a single screw, which generally has a simple single flighted design to achieve melting and metering of the molten material.

Twin screw extruders (TSEs) were developed to overcome the poor mixing performance of single screw extruders by using two screws, usually arranged side by side, rotating in the same (co-rotating) or opposing (counter-rotating) directions. Screws are typically designed to be closely or fully intermeshing, i.e. the flight tips of each screw reach the root of the opposing screw, with the exception of mechanical clearance. This allows a high degree of mixing in the ‘intermesh’ region between the two screws. TSEs operate by forced conveyance rather than relying on viscous drag flow. TSEs have the added advantage of a self-wiping action of the screws causing the extruder to be more sanitary, with less stagnation than single screw designs. TSE screws normally consist of hexagonal shafts on which interchangeable screw elements are arranged. This allows for a high degree of flexibility in screw design, which can be readily configured to provide a mixture of conveyance, kneading, mixing and venting, depending upon the application. TSEs are typically starve-fed and run with incompletely filled channels.

Counter-rotating extruders have lower levels of mixing but high material feed and conveying characteristics due to the material movement within the extruder. If the flights of each screw match and completely fill the channels of the other screw the material is completely prevented from rotating with the screw and thus positively moved in the axial direction. This movement is independent of material viscosity and adherence to the metal surfaces of the barrel and screw. Residence times and melt temperatures in counter-rotating TSEs are very uniform. Material between the screws is subjected to high shear forces and causes the development of high pressures, thus counter-rotating TSEs are operated at lower screw speeds than co-rotating due to the high pressures developed between the screws. Typical polymeric applications of counter-rotating TSEs include materials which are sensitive to thermal degradation and require low residence times such as PVC and wood composite polymers.

Co-rotating extruders are the most industrially significant class of TSE and tend to have closely or fully intermeshing screw designs. Screw elements are self-wiping and high screw speeds and throughputs are possible with this design. Co-rotating TSEs have the ability to mix the material longitudinally as well as transversely, so material is transported from one chamber of the screw to the other, which results in excellent mixing and a high input of energy into the mixture. Co-rotating screws offer a high degree of flexibility compared to counter rotating systems. Typical configurations include a mixture of conveying, kneading and mixing elements. Barrier elements can be used to provide melt seals and regions of high and low pressure to allow injection of liquids or removal of volatiles. Typical applications of co-rotating TSE include the vast majority of plastics compounding operations, where polymeric resins are mixed with a wide range of reinforcing fillers and additives. Blending and reactive extrusion are also widely used applications. Extrudate from co-rotating TSEs is generally pelletised for use in a subsequent forming process; TSE alone is not particularly well suited to manufacture of a product due to the low head pressures generated and the inherent fluctuations in output.

Widespread industrial use of extruders has conventionally been in the plastics, rubber and food industries. In recent times the potential of extrusion has begun to be realised in pharmaceutical applications, largely because a number of functions can be performed in a single continuous operation. Therefore processes conventionally carried out by a number of separate batch operations can be combined, increasing manufacturing efficiency and potentially improving product consistency. However, extrusion based pharmaceutical process design has been developed from conventional plastics processing operations in conjunction with specialist feeding and downstream handling technology—it involves the dispersion of API into a polymeric matrix in a variety of forms. Most conventional polymer processing machinery can be adapted for use in a Good Manufacturing Practices (GMP) environment. Extrusion processing operations can be readily scaled from the laboratory to manufacturing scale and lend themselves well to in-process monitoring techniques, known within the pharmaceutical industry as Process Analytical Technology (PAT).

Any conventional extruder may be used provided that it can be adapted to operate within the necessary very precise extrusion temperature range and high shear mixing needed to ensure effective conversion.

Examples of pharmaceutical extrusion applications are briefly listed below:

Solid dispersions are defined as intimate mixtures of active drug substances (solutes) and diluents or carriers (solvent or continuous phase). In conventional technologies solid dispersions of drugs are typically produced by melt or solvent evaporation methods, where the materials produced are subsequently pulverised, sieved and mixed with excipients, before being encapsulated or compressed into tablets. Melt extrusion offers an improvement in manufacture of these systems, and can be used for particulate and molecular dispersions.

Controlled-release drug delivery systems offer numerous benefits over traditional dosage forms. The most common processes for the manufacture of controlled-release tablets include wet granulation and direct compression techniques, both of which are subject to content uniformity and segregation problems. Melt extrusion technology facilitates the design and development of controlled-release oral dosage forms without the use of water or solvents. Single or twin screw extruders with downstream micropelletisation or spheronisation capability are used to produce granules or extruded tablets. Hydrophillic and hydrophobic materials, such as drugs, polymers and additives can be processed and only one component must melt or soften to facilitate material flow.

### BRIEF DESCRIPTION OF THE DRAWINGS

Embodiments of the invention are further described hereinafter with reference to the accompanying drawings, in which:

FIG. 1 illustrates screw configuration.

FIG. 2. illustrates the powder x-ray diffraction (PXRD) pattern of the pure starting material relating to orthorhombic form of artemisinin, characteristic peak at 7.89° 2θ.

FIG. 3. shows PXRD pattern of extruded artemisinin showing characteristic peak of triclinic form at 9.45° 2θ.

FIG. 4. shows PXRD pattern of orthorhombic artemisinin adapted from the Cambridge crystallographic database.

FIG. 5. shows PXRD pattern of triclinic artemisinin adapted from the Cambridge crystallographic database.

FIG. 6. shows PXRD pattern of the obtained extruded artemisinin after 3 months showing characteristic peak of triclinic form at 9.45° 2θ.

FIG. 7. shows PXRD pattern of the obtained extruded artemisinin after 6 months showing characteristic peak of triclinic form at 9.45° 2θ.

FIG. 8. shows PXRD pattern of the obtained extruded artemisinin after 9 months showing characteristic peak of triclinic form at 9.45° 2θ.

FIG. 9. shows PXRD pattern of the obtained extruded artemisinin after 12 months showing characteristic peak of triclinic form at 9.45° 2θ.

FIG. 10. shows PXRD pattern of extruded artemisinin at low temperature (T=120° C.) indicating no transformation to triclinic polymorph.

FIG. 11. shows PXRD pattern of extruded artemisinin at low shear indicating no transformation to triclinic polymorph.

FIG. 12. shows the differential scanning calorimetry (DSC) thermogram of orthorhombic artemisinin exhibiting two endothermic peaks major at 154.85° C.

FIG. 13. shows the DSC thermogram of extruded artemisinin exhibiting one melting endotherm at 155° C.

FIG. 14. shows Fourier transform infrared spectroscopy (FTIR) spectrogram of orthorhombic artemisinin.

FIG. 15. shows FTIR spectrogram of extruded artemisinin where the IR spectra for triclinic is significantly broader than orthorhombic at the region between 2845-3000 cm-1 and 1300-1500 cm-1.

FIG. 16. shows dissolution profile of the pure starting material and extruded artemisinin.

FIG. 17. shows nuclear magnetic resonance (NMR) spectrum of the pure starting material depicting 1H-NMR signal at 5.864.

FIG. 18. shows NMR spectrum of extruded artemisinin showing 1H-NMR signal at 5.876.

FIG. 19. shows high-performance liquid chromatography mass spectroscopy (HLPC-MS) chromatogram of orthorhombic artemisinin.

FIG. 20. shows HLPC-MS spectrum of extruded artemisinin matching the spectrum of the pure starting material.

FIG. 26. shows PXRD pattern of triclinic form obtained from recrystallization.

FIG. 27. shows PXRD pattern of recrystallised triclinic form after a week.

FIG. 28. shows PXRD pattern of the pure starting chlorpropamide form A showing characteristic peak at 6.97° 2θ.

FIG. 29. shows PXRD pattern of the extruded material: showing characteristic peak of chlorpropamide form C at 13.88° 2θ.

FIG. 30. shows PXRD pattern of the pure starting material of monoclinic carbamazepine showing characteristic peak at 15.36° 2θ.

FIG. 31. shows PXRD pattern of extruded material showing characteristic peak at 7.92° 2θ of triclinic carbamazepine.

FIG. 32. shows PXRD pattern of the pure starting material of piracetam form III showing characteristic peak at 14.91° 2θ.

FIG. 33. shows PXRD pattern of the extruded material showing characteristic peak of piracetam form I at 12.96° 2θ.

FIG. 34. shows a calibration curve for artemisinin.

FIG. 35 shows the plasma concentration profiles of the orthorhombic and the triclinic forms of artemisinin.

### DETAILED DESCRIPTION

When certain substances are subject to temperature and pressure in combination, by processing the substance within a heated extruder, and therefore exposing the substance to a sustained process of shear and temperature, the substance can transform into a metastable polymorph. The inventors have surprisingly identified that when the substance is extruded at a temperature between around 8° C. to 20° C. below the substance's melting point, polymorphic transformation of the substance to a metastable polymorph may occur. In other words, the inventors have surprisingly generated solvent free, metastable polymorphs of certain pharmaceutical products.

Advantageously, the present method is continuous and does not suffer from the problems associated with batch processing such as limitations of scale up, purity, but most problematic, issues with stability. The process of the present invention is simple to scale up, continuous, solvent free, whereby the resultant processed substances have high purity and stability compared to traditional solvent crystallisation techniques and other processes noted above.

The method can be used to provide solvent free stabilised metastable form

The present invention is a new solvent free continuous technology for the generation of a metastable polymorph using screw extrusion where appropriate temperature and shear cause transformation to occur. The metastable form obtained using our method is more stable as compared to the conventional solvent crystallisation technique. It is practically promising, scalable, reproducible, high yield, single step technique to obtain metastable polymorphs for drugs which require polymorphs transformation for efficacy. This novel approach is of interest of from the both perspective high throughput and green chemistry regulation.

The inventors have successfully demonstrated transformation in four drug molecules including, artemisinin, piracetam, carbamazepine and chlorpropamide. However, it should be noted that the present invention has application beyond the drug molecules noted above. This method may be applicable for other pharmaceutical drugs where the metastable form is more efficient.

### General Experimental Methodology

A co-rotating twin screw extruder was used in the formation of polymorphs, having a screw diameter of 16 mm. An extruder with L:D ratio of 40:1 (Thermo Prism Eurolab) was also used, incorporating a total of 10 temperature controlled barrel and die zones. Extruder length combined with screw design determines the residence time and the degree of mixing possible during extrusion.

### Experimental Procedure

A cleaned extruder was pre-heated to the selected processing temperature. A range of barrel temperature profiles were used, typically increasing from a cooled feed zone to a maximum along the barrel towards the die end. For the purposes of these trials the extruder was run without a die. Extruder screw rotation speed was set; a wide range of speeds can be achieved, up to 200 revolutions per minute (rpm) with the extruders used here. Typical screw rotation speeds were set at between 5 and 25 rpm. The substance was then introduced into the feed hopper of the extruder, here for small batch sizes (typically between 10-30 g) feedstock was manually dosed using a spatula. For larger batch sizes a gravimetric feeder system was employed. The extruded product was then collected at the exit of the screws, in powder form. The collected product was cooled to room temperature and subsequently analysed using an X-ray diffractometer (Bruker D8). Further characterisation of the collected product was performed using differential scanning calorimetry (DSC), Fourier transform infrared spectroscopy (FT-IR), dissolution studies, nuclear magnetic resonance (NMR), and high performance liquid chromatography mass spectroscopy (HLPC-MS).

During the course of experiments, the following parameters could be adjusted: set temperature, screw rotation speed, throughput, screw design (i.e. degree of distributive and dispersive mixing), and the number of passes through the extruder

As noted above, the inventors have successfully demonstrated transformation in four drug molecules including, artemisinin, piracetam, carbamazepine and chlorpropamide. Below, examples of the experimental parameters and results are provided for each of the above drug molecules.

### Artemisinin

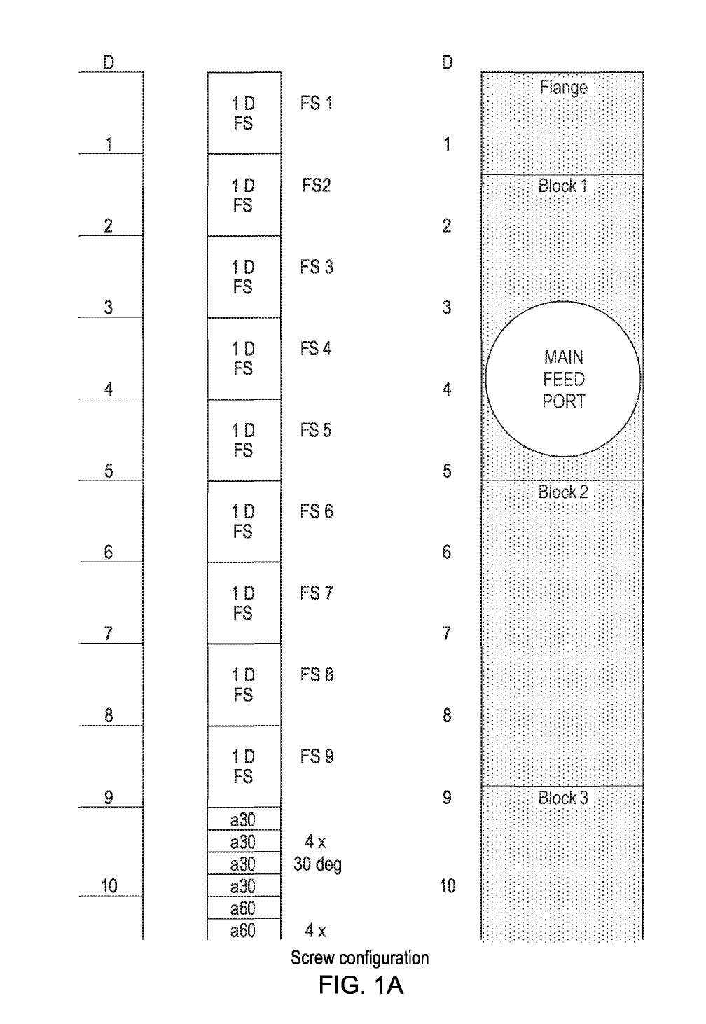

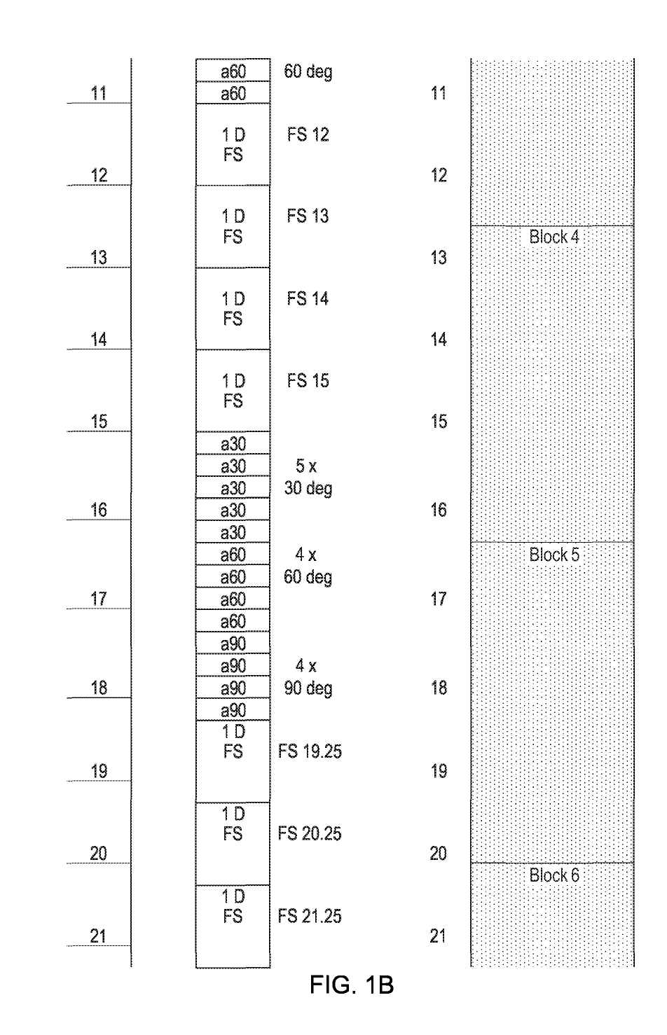

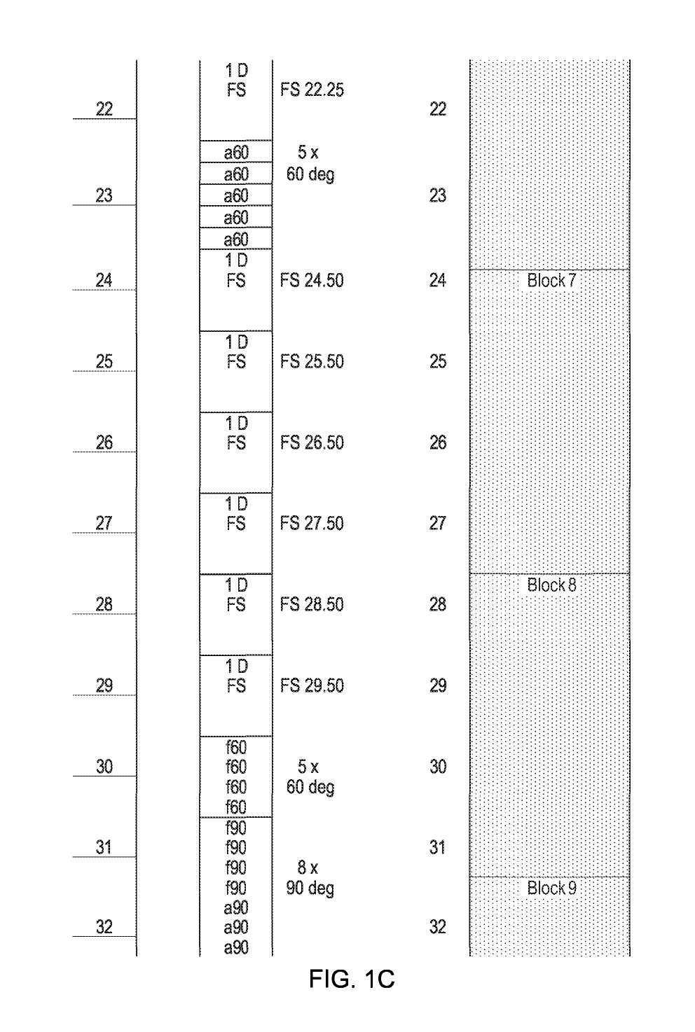

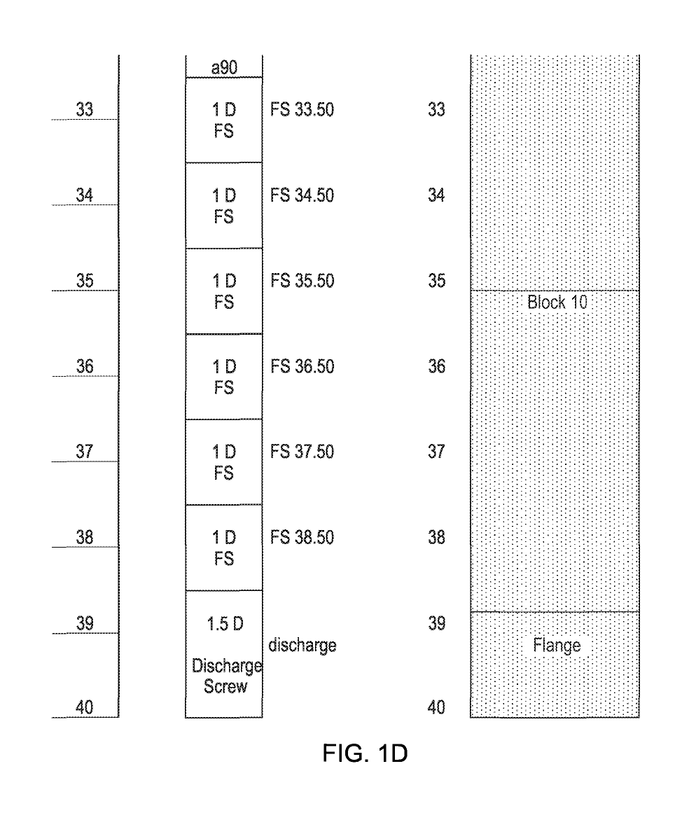

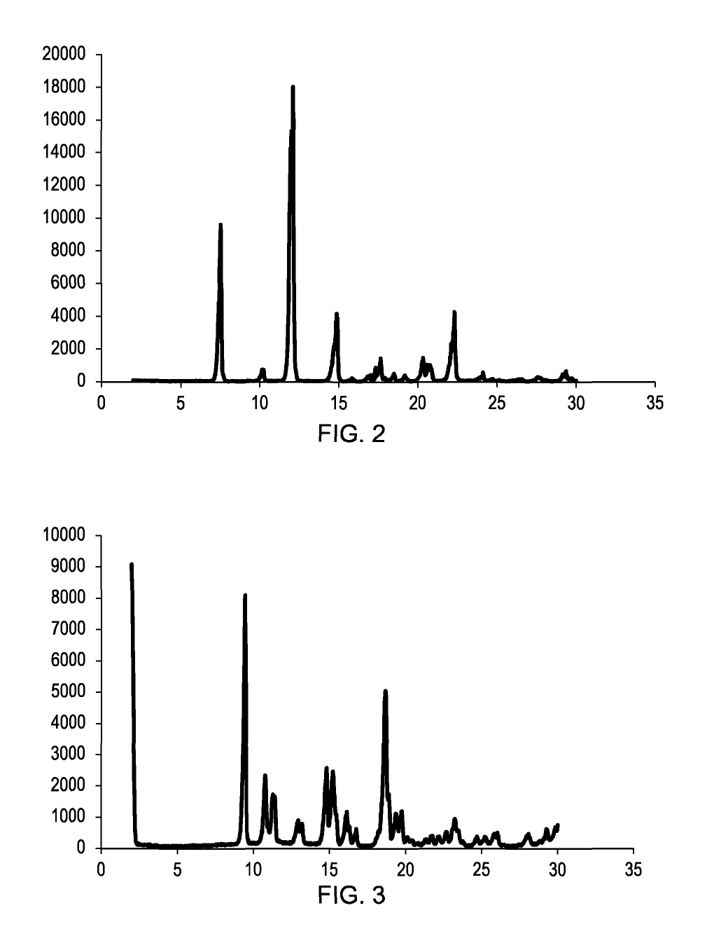

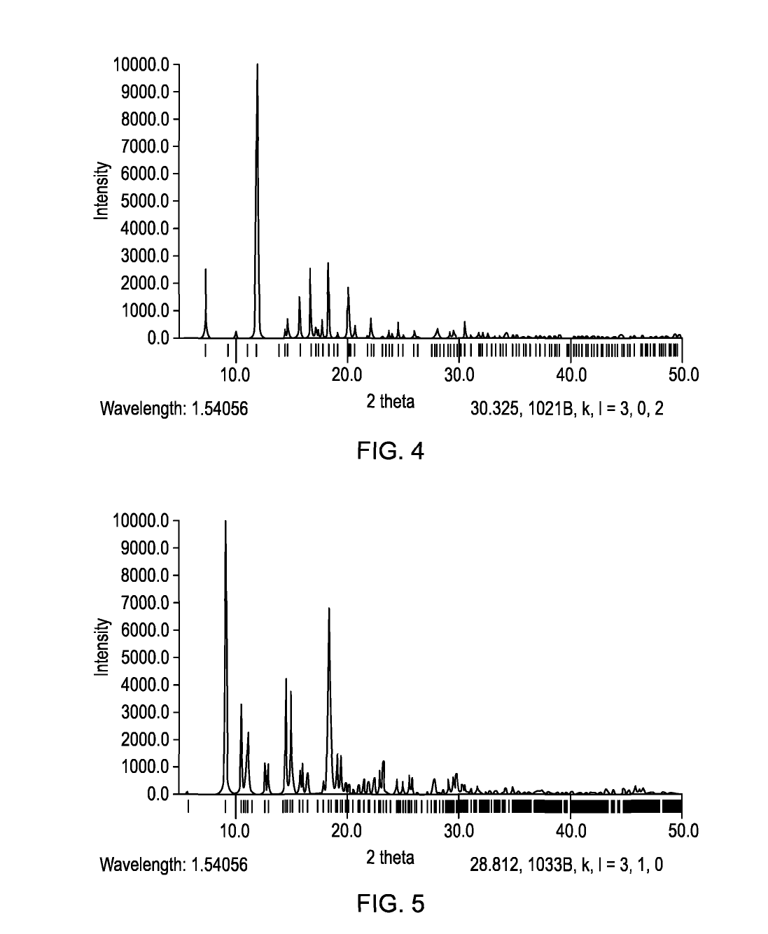

Extrusion was carried out using a 16mm twin screw extruder (pharmalab, thermo scientific, UK) with L:D ratio 40:1. Barrel temperature was set to T145 (see Table 1) and allowed to stabilise the temperature for 15 minutes. One hundred grams of artemisinin (orthorhombic form) was fed at 3 grams per min feed rate and screw speed was 20 rpm. The twin screw configuration is displayed in FIG. 1 and temperature profile T 145 is displayed in Table 1. The residence time was 12 min and the product was collected at the discharge screw. The obtained product was cooled to room temperature and crystalline patterns was examined using a Bruker D8 (wavelength of X-ray 0.154 nm Cu source, voltage 40 kV and filament emission 40 mA). FIG. 3 illustrates the PXRD patter of the extruded artemisinin. The formation of triclinic was identified from characteristic PXRD peak at 9.45° 2θ. FIG. 3 should be compared with FIG. 2 which illustrates the PXRD pattern of pure artemisinin before being extruded. The starting substance belongs to the orthorhombic form of artemisinin with a characteristic peak at 7.89° 2θ. For comparative purposes, FIGS. 4 and 5 show the PXRD pattern of orthorhombic and triclinic artemisinin respectively, adapted from the Cambridge crystallographic database.

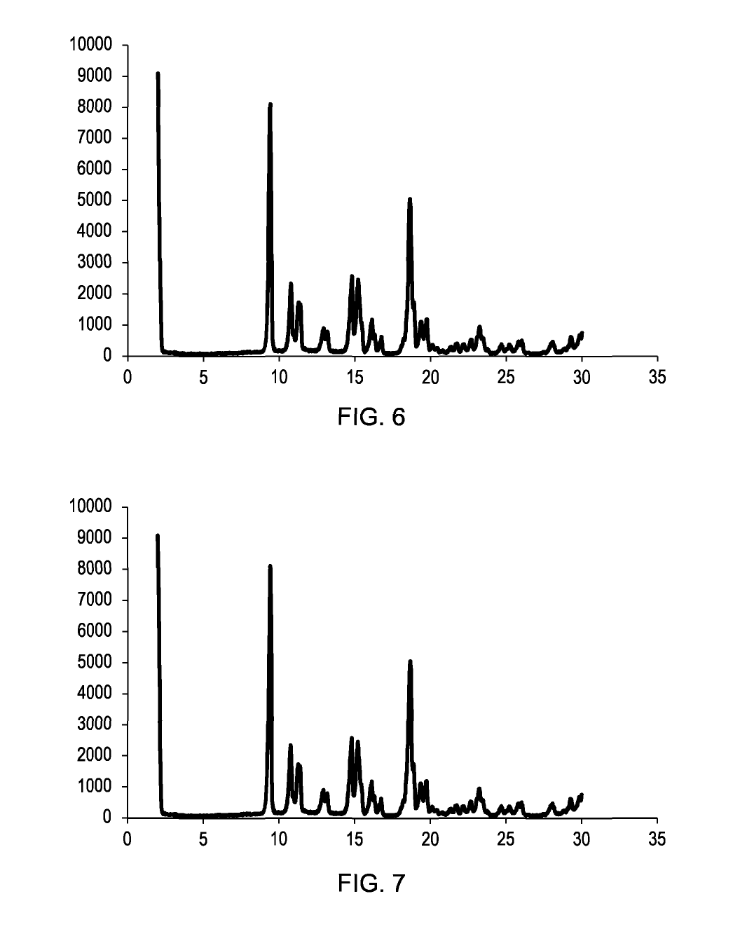

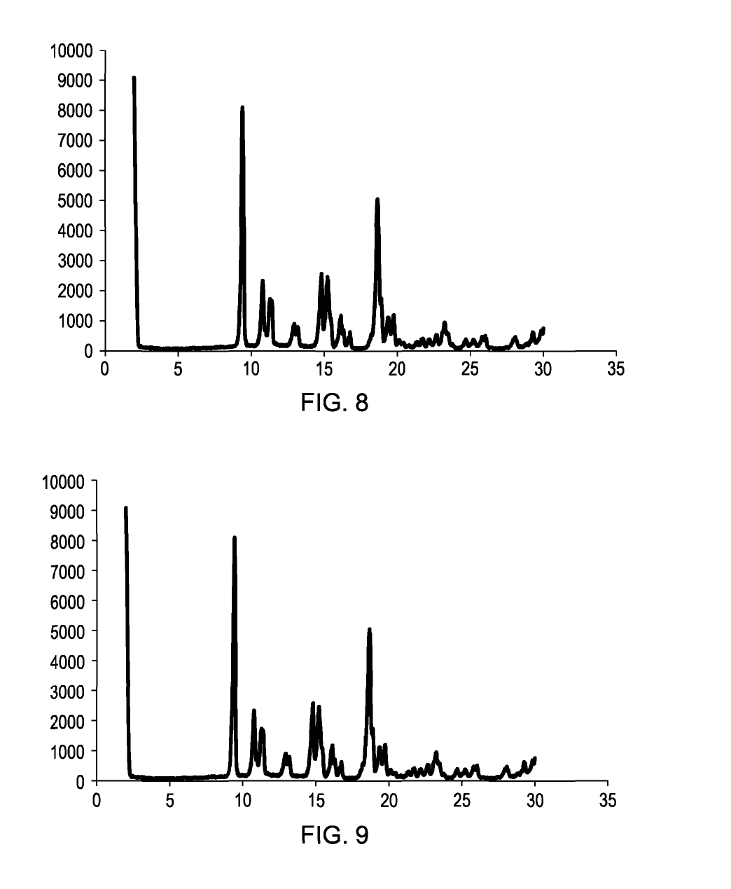

Powder x-ray diffraction was used to assess the long term stability of the extruded artemisinin. FIG. 6 shows the PXRD pattern for extruded artemisinin after three months exhibiting a characteristic peak of triclinic form at 9.45° 2θ. FIG. 7 shows the PXRD pattern for extruded artemisinin after six months exhibiting a characteristic peak of triclinic form at 9.45° 2θ. FIG. 8 shows the PXRD pattern for extruded artemisinin after nine months exhibiting a characteristic peak of triclinic form at 9.45° 2θ. FIG. 9 shows the PXRD pattern for extruded artemisinin after twelve months exhibiting a characteristic peak of triclinic form at 9.45° 2θ. The PXRD patterns highlight the remarkable stability of the extruded artemisinin measured at regular intervals over a twelve month period. FIG. 36 shows the PXRD pattern for extruded artemisinin after 24 months exhibiting a characteristic peak of triclinic form at 9.45° 2θ confirming stability of the extruded artemisinin over a 24 month period. This result highlights the remarkable potential shelf-life of the extruded artemisinin.

The triclinic form was also produced with temperature profile T140. The temperature profile T 140 is displayed in Table 1A. The product obtained using temperature profile T140 was pure triclinic form and the properties and stabilities of the pure triclinic form were the same as that obtained using temperature profile T145 described above.

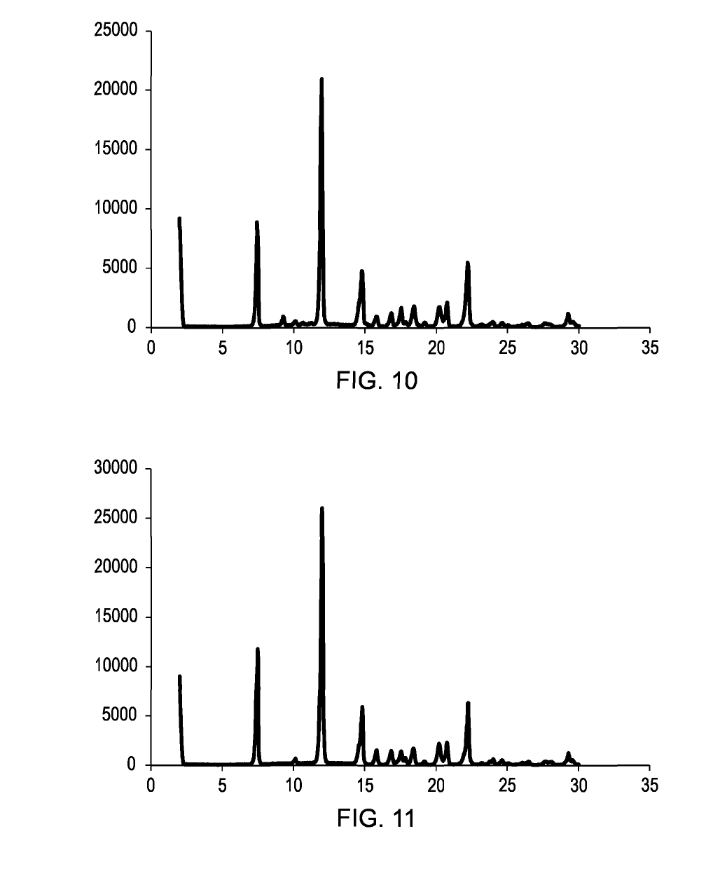

FIGS. 10 and 11 illustrate the importance of the specified temperature and shear ranges. FIG. 10 shows the effect of extruding artemisinin at low temperatures. FIG. 11 shows the effect of extruding artemisinin at low shear. Neither indicate any evidence of polymorphic transformation to triclinic form.

The extruded artemisinin was further characterised by DSC, FT-IR, Dissolution, NMR and HPLC-MS.

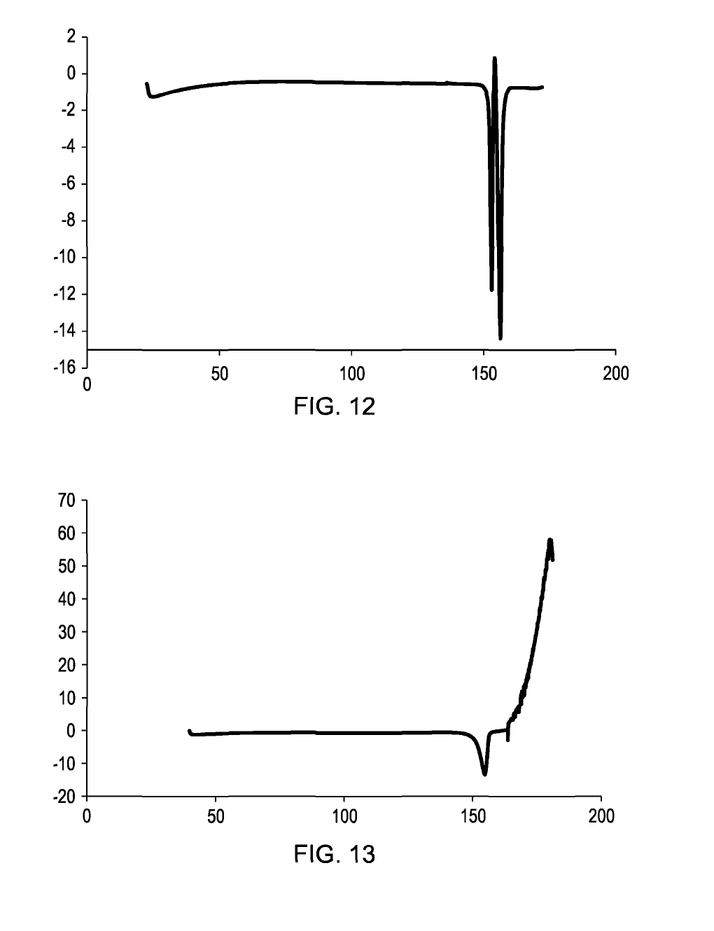

Thermal behaviour of samples was characterised by DSC scanning in the range 25 to 175° C. using instrument TA Q2000 along with RCS90 cooling unit. The temperature calibration was done using indium metal in the range 25 to 200° C. Approximately 3mg of sample was weighed and placed into an aluminium pan while the empty aluminium pan was used as a reference. The analysis was executed under cooling rate 10° Cmin⁻¹ and the nitrogen flow rate was 50 ml/min to maintain an inert environment. FIG. 12 illustrates the DSC thermogram of orthorhombic artemisinin. The thermogram exhibits two endothermic peaks majoring at 154.85° C. FIG. 13 shows the thermogram of extruded artemisinin. Here, one melting endotherm can be observed at 155° C.

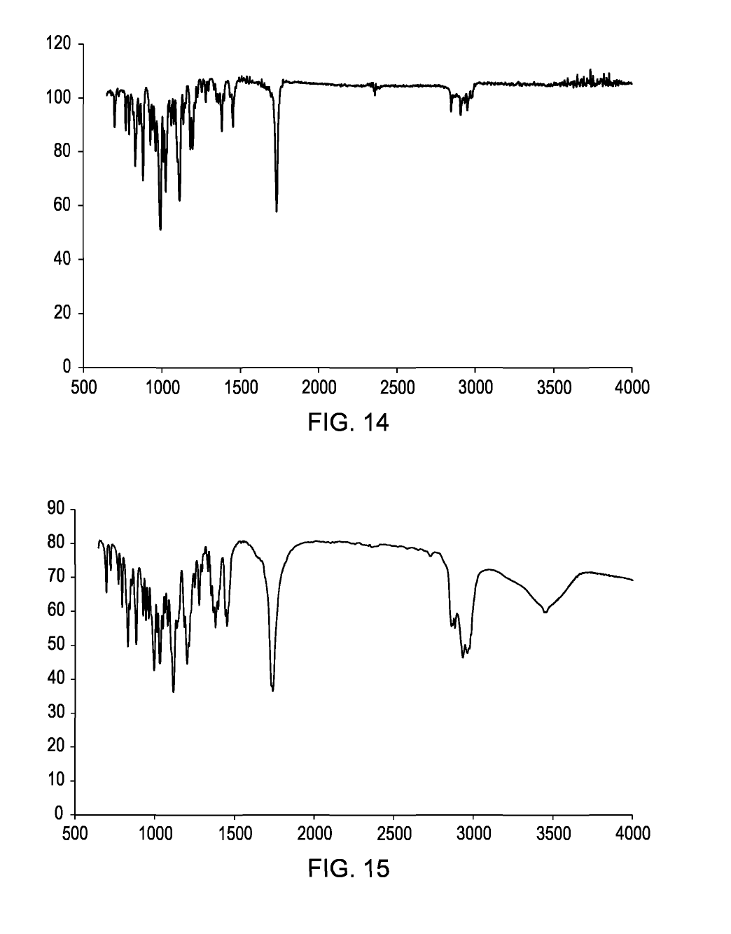

For FT-IR studies, artemisinin crystals were diluted by up to 1% using KBr. Artemisinin and KBr were triturated and mixed carefully using mortar and pestle. This mixture was transferred in between two stainless steel disc dies, then compressed at about 9 tons through a hydraulic press to form a uniform disc. The IR spectrum of this disc sample was displayed by infrared beam irradiation from light source Glowbar at 4 cm⁻¹ resolution and at 20 scans using Bomen Fourier Transform Infrared, Model. FIG. 14 shows the FT-IR spectrogram of orthorhombic artemisinin. FIG. 15 shows the FT-IR spectrogram of extruded artemisinin. The IR spectra for triclinic form is significantly broader than orthorhombic form at the region between 2845-3000 cm⁻¹ and 1300-1500 cm⁻¹.

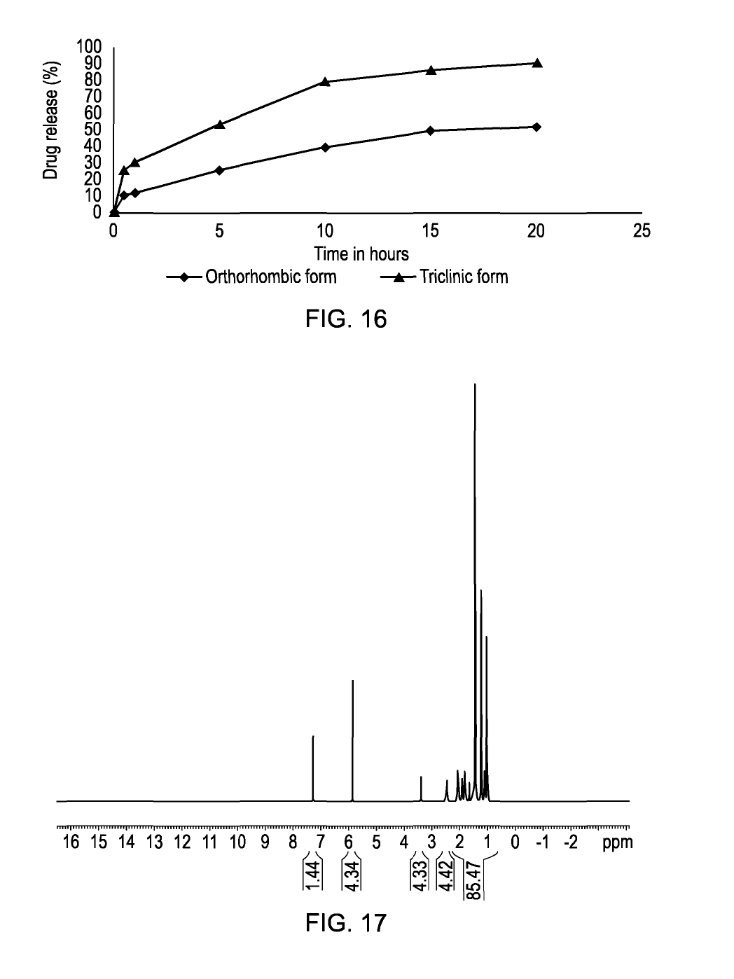

In-vitro dissolution profile was studied by USP-XXVI paddle method using dissolution test apparatus (Copley Scientific, Nottingham, UK). Drug release from processed artemisinin was compared with pure artemisinin and the results shown in FIG. 16. Water was used as the dissolution medium. The experiment was performed at 75 rpm in 600 ml medium at 37° C.+0.1° C. At predetermined time intervals, 5 ml of sample was taken and replaced with the same volume of fresh medium. The collected sample was filtered using a cellulose acetate filter. 20 mg of artemisinin was used for the dissolution study. 1 ml of sample was treated with alkali reaction by adding 2 ml of 0.2% NaOH and heated in water bath at 50° C. for 30 minutes and UV absorbance was detected at 290 nm. PCP disso software V3 (Poona College of Pharmacy, Pune, India) was used to calculate per cent release of drug. Extruded crystals showed four times greater dissolution rate in comparison with starting material.

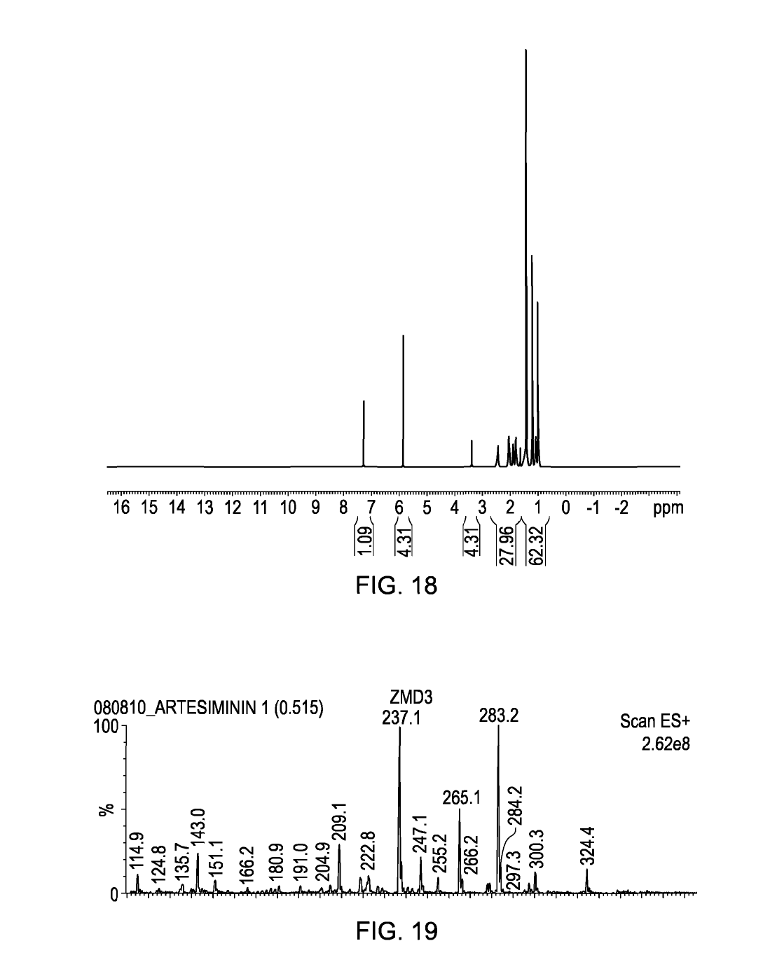

NMR analysis was carried out using BrukerAvance-II 500 MHz NMR spectrometer equipped with 1H-detection. Accurately weighed 1.8 mg of pure artemisinin and processed artemisinin was dissolved in CDCl3 solvent. FIGS. 17 and 18 compare the NMR spectrum of the pure starting material with the extruded material. The pure starting material exhibits 1H-NMR signal at 5.864 whereas the NMR spectrum of extruded artemisinin shows 1H-NMR signal at 5.876.

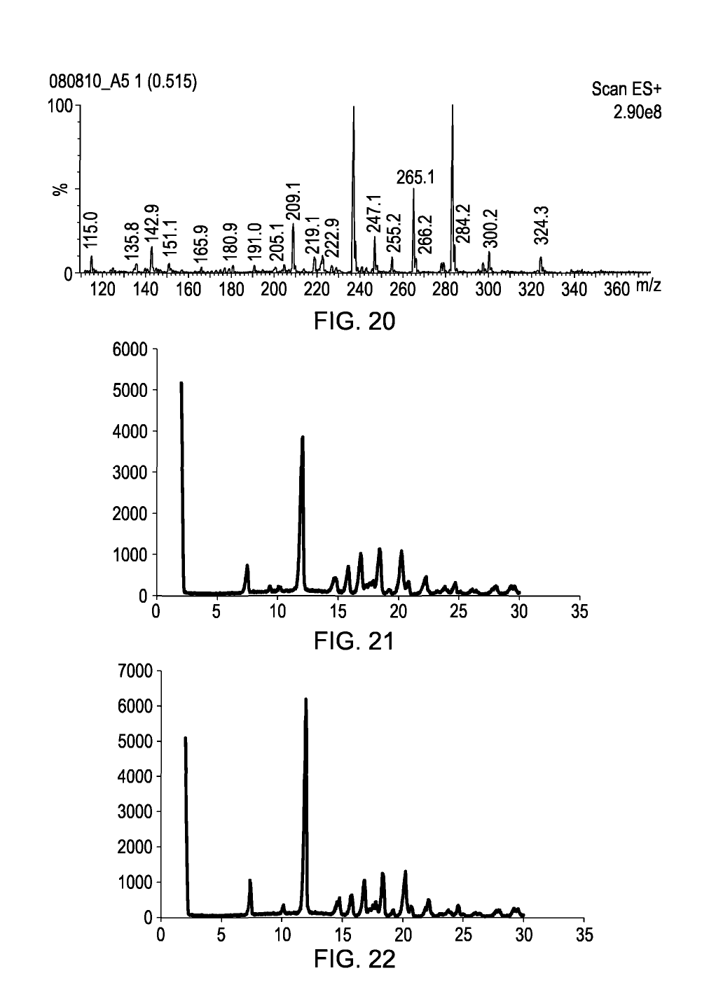

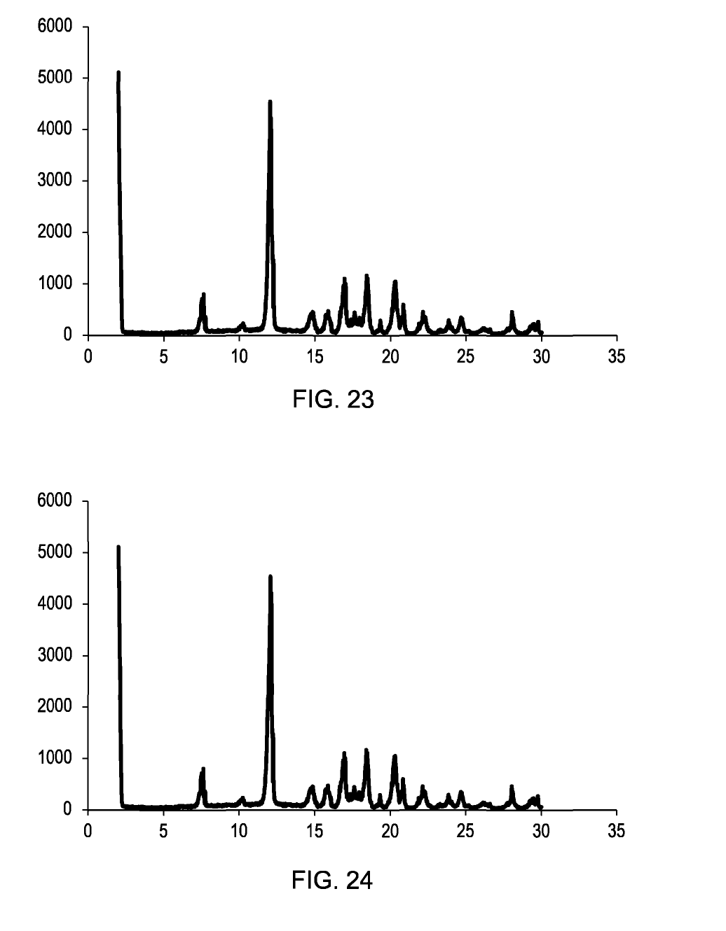

HPLC was performed using a Waters Alliance separation module 2695. Column C18, 3×100 mm, and 1.8 um particle size was used and 1 ul of artemisinin was loaded. 50% acetonitrile, 50% water, 0.09% formic acid and 0.01% trifluroacetic acid was used as a mobile phase. FIG. 19 shows HPLC-MS chromatogram of orthorhombic artemisinin exhibiting a high resolution of mass spectrum at 283.2 ion corresponding to the molecular formula C15 H23 O5. Additional peaks were obtained at 324.4, 265.1, 237.1 and 300.3 related to [M+Na], loss of water, loss of water and carbon monoxide (CO) and loss of water and two CO respectively. FIG. 20 shows HLPC-MS spectrum of extruded artemisinin which corresponds with the spectrum of the pure starting material.

Effect of different solvents such as acetone, ethanol, cyclohexane, methanol and water on extruded triclinic form was studied. In 3 g of extruded sample 0.2 ml of solvent was added separately and stability was evaluated by PXRD. FIGS. 21 to 25 show the PXRD patterns highlighting the effect of adding the different solvents to extrude. It was observed that conversion rate from triclinic to orthorhombic form was proportional to solubility of orthorhombic form in each solvent. The rank order of transformation (displayed in table 4) is acetone>methanol>ethanol>cyclohexane>water. In the sample containing water no transformation was observed because the orthorhombic form has low solubility in water. Table 4 shows the stability of extruded artemisinin in the presence of externally added solvents.

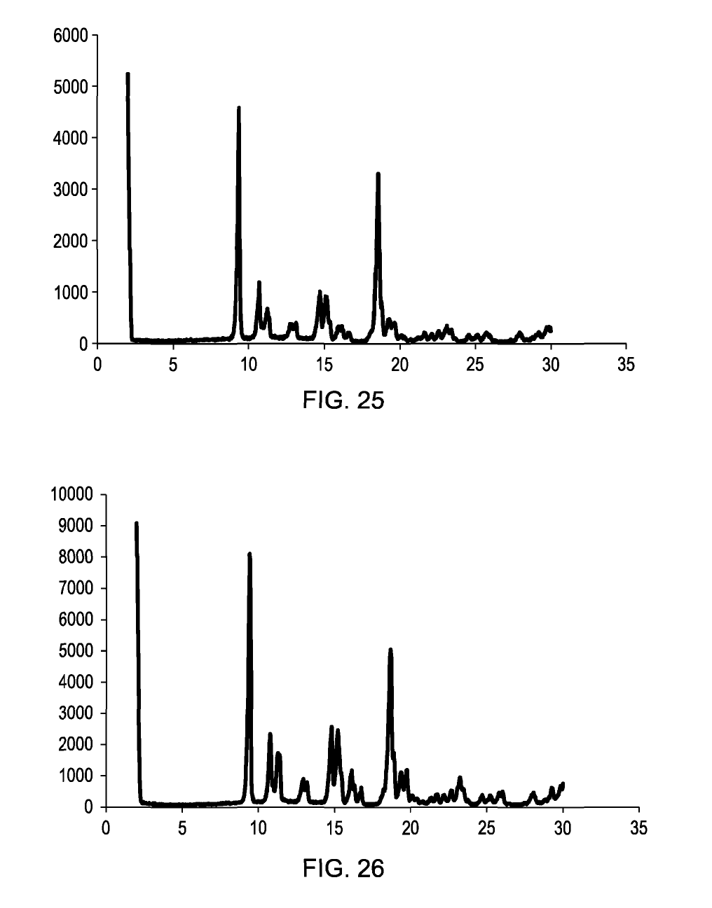

The triclinic polymorph of artemisinin was prepared by recrystallisation from cyclohexane at 80° C. The product obtained was vacuum dried and the crystal form was confirmed by PXRD. Stability study was performed and after a week triclinic form was transformed into more stable orthorhombic form. FIG. 26 shows PXRD pattern of triclinic form obtained from recrystallisation.

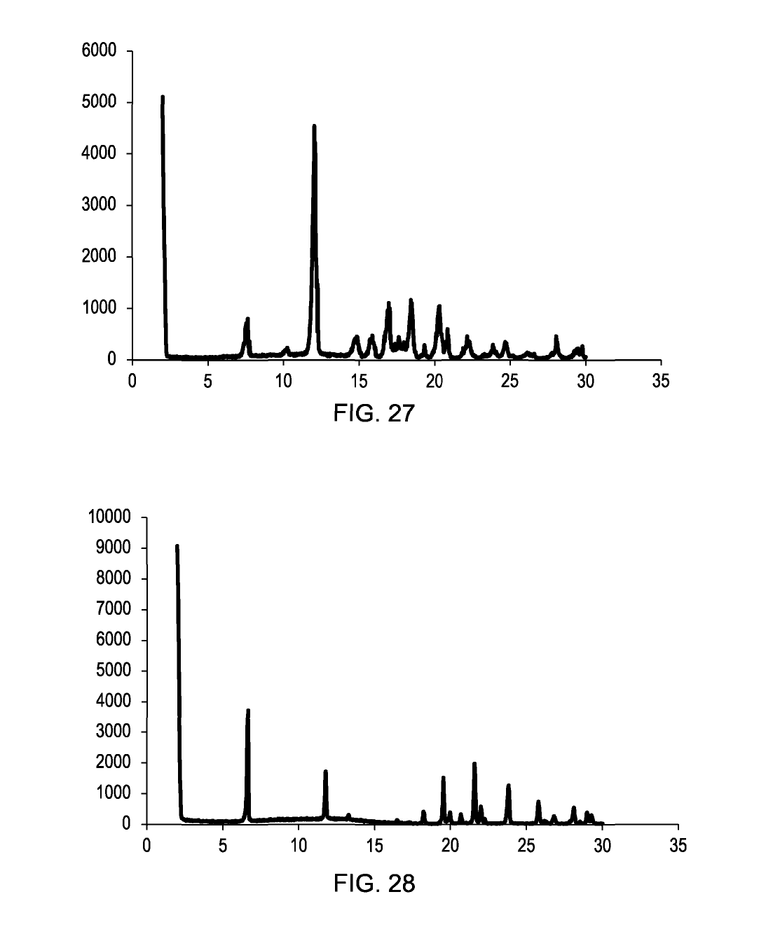

The triclinic form prepared from solvent crystallisation transformed to orthorhombic form within a week. FIG. 27 shows PXRD pattern of recrystallised triclinic form after a week. The triclinic form prepared from solvent crystallisation transformed to orthorhombic form within a week.

A pharmacokinetic study of artemisinin was carried out. The study was performed using 36 healthy albino wistar rats with a weight ranging from 180 to 200 grams. The wistar rats were taken and divided into three groups; a control group, the orthorhombic crystal form group and the triclinic crystal form group. A sparse technique was used to collect blood samples (n=6). The animals were housed in standard metabolism cages and were subject to fasting conditions for 12 hours before dosing. The animals were allowed free movement and access to water throughout the experiment. 100 milligrams of artemisinin was dispersed in 0.5% aqueous carboxymethylcellulose (CMC) solution. The oral dose (equivalent to 100 mg of artemisinin) was administered using an oral syringe. At predetermined time intervals, blood samples were obtained by the retro orbital technique and collected in EDTA tubes.

Plasma was obtained by centrifugation of the blood sample at 3500 revolutions per minute (rpm) for 15 minutes. A volume of 200 μl of plasma was pipetted into Eppendorf tubes and 100 μl of internal standard (artemether solution 1000 μl/ml) and 700 μl methanol were added. The solution was vortexed for 2 minutes and the organic phase was separated by centrifugation. The collected sample was then subjected to analysis by HPLC. The plasma level of artemisinin was analysed by HPLC using 65% acetonitrile and 35% water as the mobile phase. The HPLC system consisted of an Agilent 1200 series, UV detector (Agilent Technologies, IQ Winnersh, Wokingham, United Kingdom) set at 210 nm and a C18 column (250×4.6 mm). Artemisinin exhibits a maximum UV absorption at 210 nm. The limit of detection and quantification were 1.01 and 3.06 μg/ml, respectively. The concentration against peak area graph plot was found to be linear (r2=0.998).

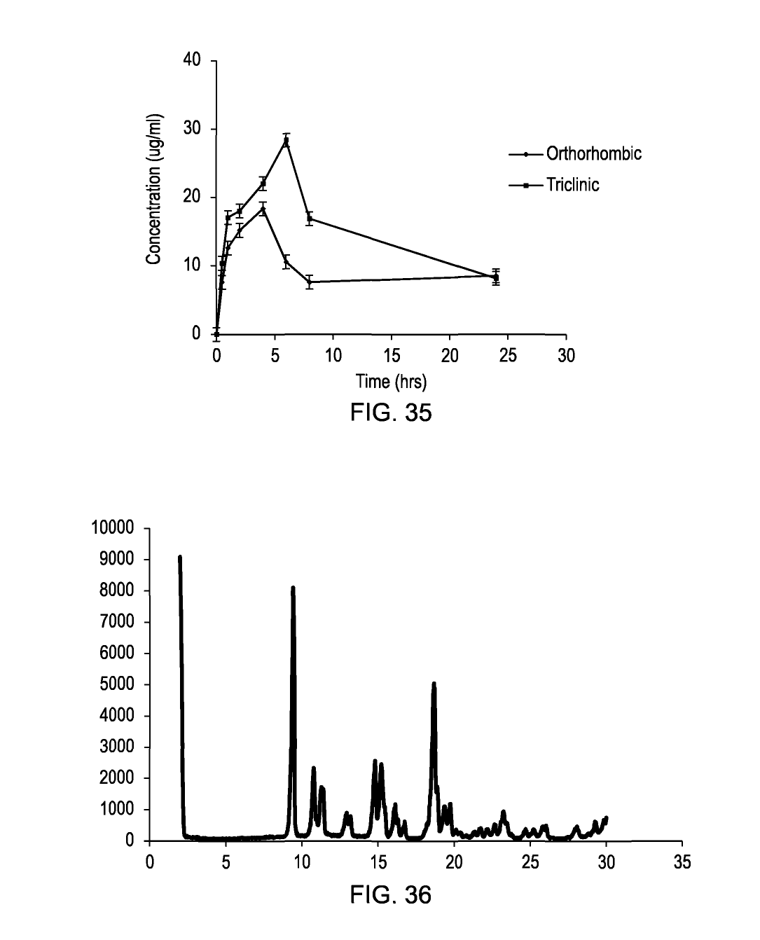

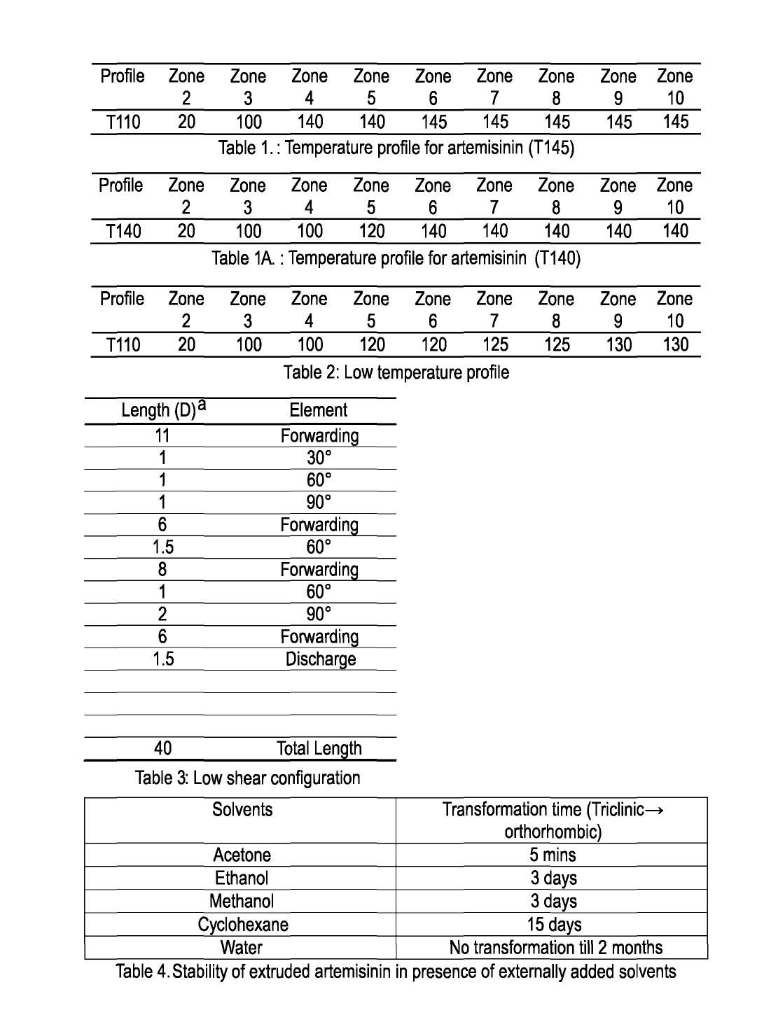

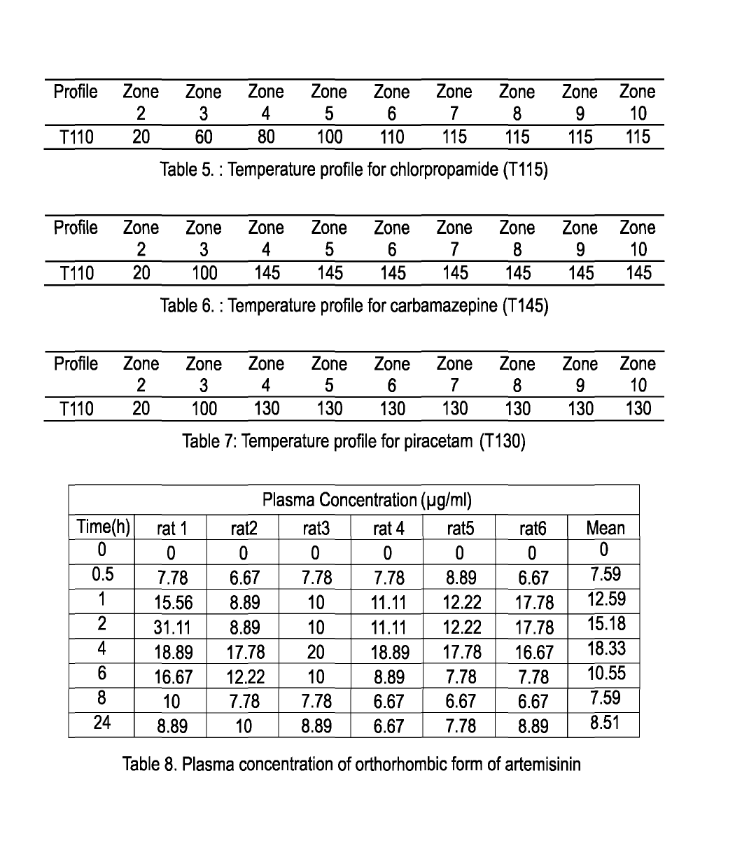

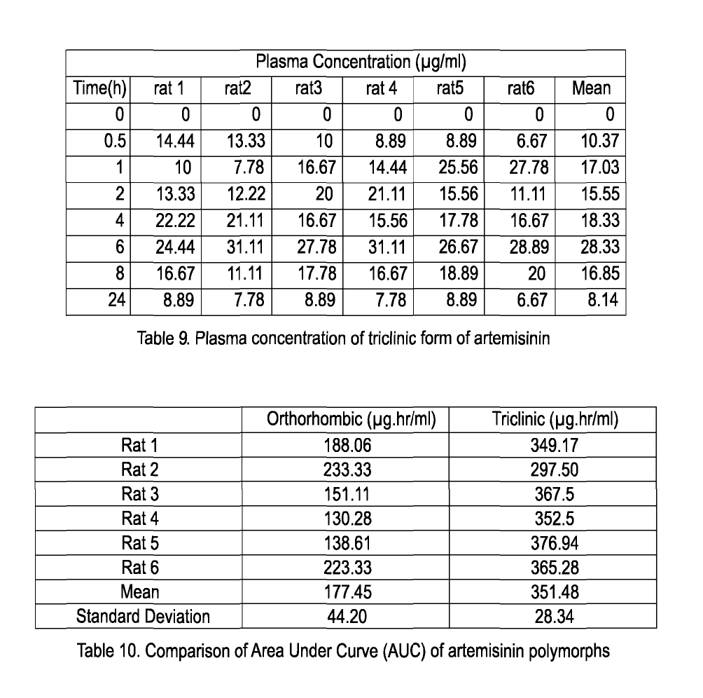

The HPLC calibration curve is shown in FIG. 34. The artemisinin plasma concentrations achieved at different times after administration of the orthorhombic and triclinic forms are given in Table 8 and Table 9 respectively. The plasma concentration-time profiles were plotted and Areas Under Curve (AUC) were calculated (shown in FIG. 35) using the Trapezoidal rule. The AUCs obtained for orthorhombic and triclinic forms are shown in Table 10.

The results of the pharmacokinetic study clearly demonstrate that the triclinic form has a two fold increase in AUC when compared with the orthorhombic form. This correlates with potentially improved bioavailability. Such an improved bioavailabilty would allow the possibility of lower dosage levels in for example human patients, for the same level of therapeutic efficacy when compared with existing therapies. It may also correlate with improved therapeutic efficacy at a give dosage when compared with standard therapies. Reduced dosage levels whilst maintaining or improving therapeutic efficacy could have potentially beneficial effects in terms of reducing unwanted side effects. The average maximum concentration (Cₘₐₓ) for orthorhombic and the triclinic forms was 16 μg/ml and 31 μg/ml respectively. The higher Cₘₐₓ of the triclinic form may again also contribute to the possibility of reduced levels of dosing compared to existing therapies. The average time where the concentration was found to reach a maximum (Tₘₐₓ) was 4 hours and 5 hours for the orthorhombic and the triclinic forms respectively.

### Chlorpropamide

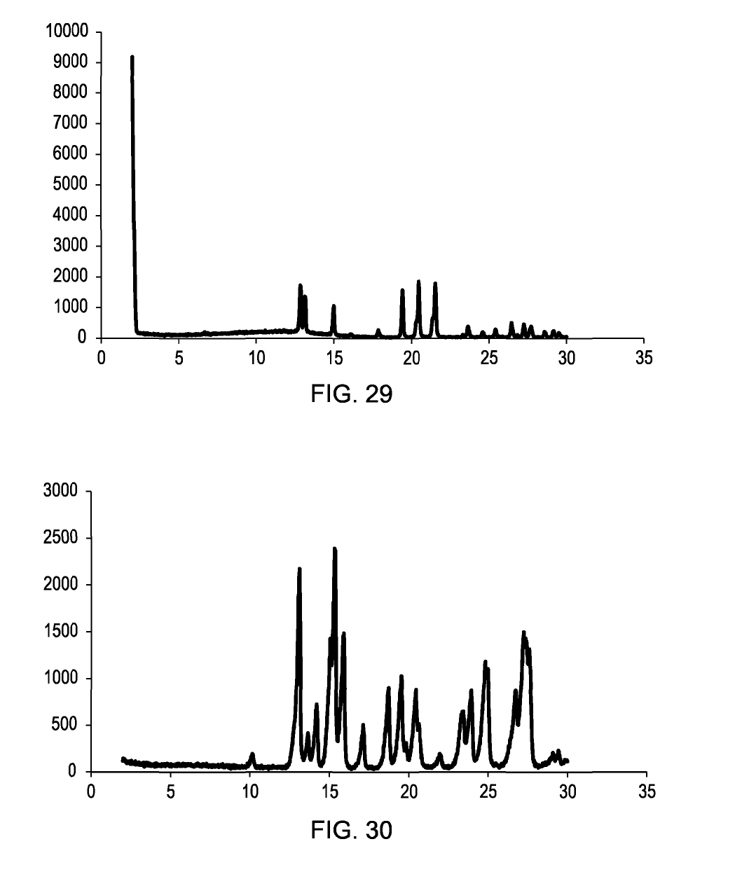

Extrusion was carried out using a 16 mm twin screw extruder (pharmalab, thermo scientific, UK) with L:D ratio 40:1. Barrel temperature was set to T115 (see Table 5) and the temperature allowed to stabilise for 15 min. One hundred grams of chlorpropamide form A was fed at 3 grams per min feed rate and screw speed was 10 rpm. The residence time was 11 min 19 sec and the product was collected. The screw configuration was showed in FIG. 1 and temperature profile T115 displayed in table 5. The obtained product was cooled to room temperature and crystalline patterns examined using a Bruker D8 (wavelength of X-ray 0.154 nm Cu source, voltage 40 kV and filament emission 40 mA). The formation of form C was identified from PXRD pattern. FIG. 28 shows PXRD pattern of the pure starting chlorpropamide form A showing characteristic peak at 6.97° 2θ. FIG. 29 shows PXRD pattern of the extruded material showing a characteristic peak of chlorpropamide form C at 13.88° 2θ.

### Carbamazepine

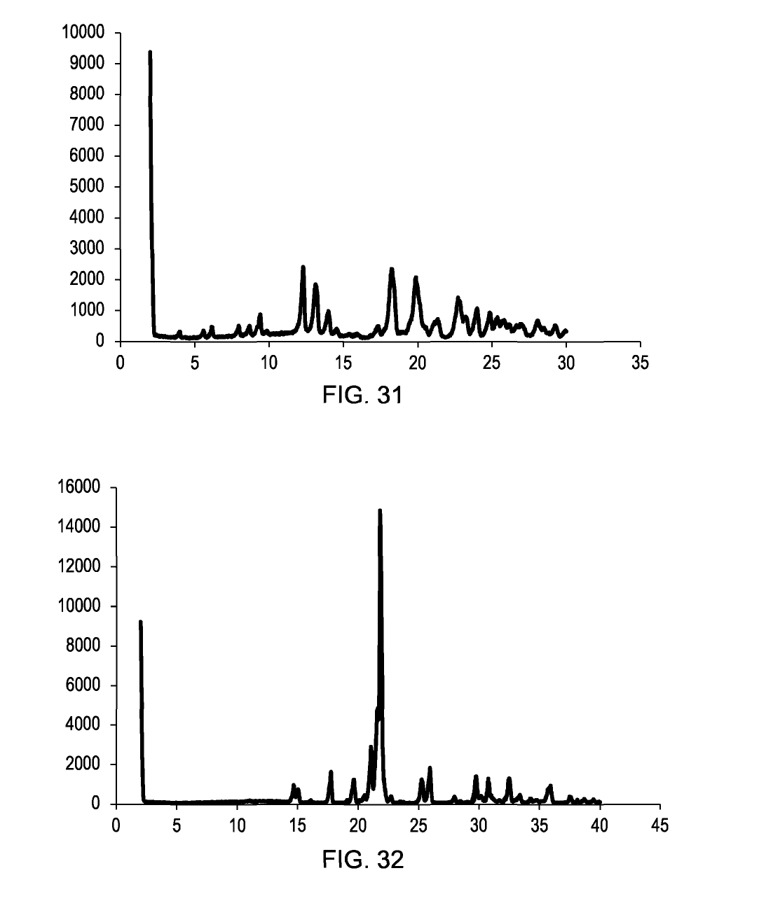

Extrusion was carried out using a 16 mm twin screw extruder (pharmalab, thermo scientific, UK) with L:D ratio 40:1. Barrel temperature was set to T145 (see Table 6) and allowed to stabilise the temperature for 15 min. One hundred grams of monoclinic form of carbamazepine was fed at 3 grams per min feed rate and screw speed was 10 rpm. The residence time was 10 min and the product was collected and reprocessed. The screw configuration was shown in FIG. 1 and temperature profile T145 displayed in table 6. The obtained product was cooled to room temperature and crystalline patterns examined using a Bruker D8 (wavelength of X-ray 0.154 nm Cu source, voltage 40 kV and filament emission 40 mA). The formation of triclinic form was identified from PXRD pattern. FIG. 30 shows the PXRD pattern of the pure starting material of monoclinic carbamazepine showing characteristic peak at 15.36° 2θ. FIG. 31 shows PXRD pattern of extruded material showing characteristic peak at 7.92° 2θ of triclinic carbamazepine.

### Piracetam

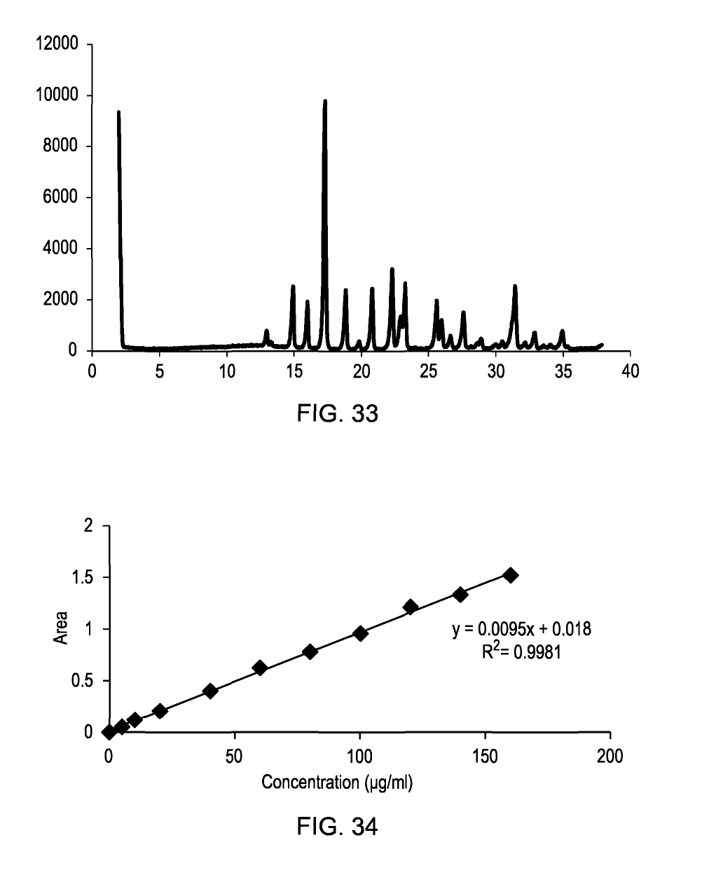

Extrusion was carried out using a 16 mm twin screw extruder (pharmalab, thermo scientific, UK) with L:D ratio 40:1. Barrel temperature was set to T130 (see Table 7) and the temperature allowed to stabilise for 15 min. One hundred grams of piracetam form III was fed at 3 grams per min feed rate and screw speed was 10 rpm. The residence time was 8 min and the product was collected. The obtained product was cooled to room temperature and crystalline patterns examined using a Bruker D8 (wavelength of X-ray 0.154 nm Cu source, voltage 40 kV and filament emission 40 mA). The screw configuration was shown in FIG. 1 and temperature profile T130 is displayed in table 7. The formation of form I was identified from PXRD pattern. FIG. 32 shows PXRD pattern of the pure starting material of piracetam form III showing characteristic peak at 14.91° 2θ. FIG. 33 shows PXRD pattern of the extruded material showing characteristic peak of piracetam form I at 12.96° 2θ.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A solvent-free method, the method comprising the steps:
(a) placing an amorphous or crystalline compound into an extruder;
(b) providing a source of heat to the extruder to heat the compound to a temperature in the range of from 8° C. below the melting point of the compound to 20° C. below the melting point of the compound; and
(c) extruding the compound from the extruder; and
(d) generating a metastable polymorph of the compound.

2. The method as claimed in claim 1, wherein the compound is an API.

3. The method as claimed in claim 1, wherein the source of heat is operative to heat the compound to a temperature in the range of from 10° C. below the melting point of the compound to 15° C. below the melting point of the compound.

4. The method as claimed in claim 1, wherein the extruded compound is held at the conversion temperature immediately after extrusion for a period of time of 30 seconds to 10 minutes.

5. The method as claimed in claim 1, wherein the method is a continuous method.

6. The method as claimed in claim 1, wherein the extruder is a twin screw extruder.

7. The method as claimed in claim 1, wherein the compound resides within the extruder for a residence time of 30 seconds to 15 minutes.

8. The method as claimed in claim 1, wherein the compound is an organic compound with a molecular weight between 100 and 600.

9. A substantially pure metastable polymorphic form of a compound having at least one thermodynamically more stable polymorphic form.

10. The substantially pure metastable polymorphic form of a compound of claim 9 having less than 2% of any other polymorphic form of the compound present as an impurity.

11. An apparatus suitable for polymorphic transformation of a substance including an extrusion device, a source of heat and a high shear mixing means.

12. The apparatus of claim 11, wherein the extrusion device is a twin screw extruder.

13. The method as claimed in claim 2, wherein the source of heat is operative to heat the compound to a temperature in the range of from 10° C. below the melting point of the compound to 15° C. below the melting point of the compound.

14. The method as claimed in claim 2, wherein the extruded compound is held at the conversion temperature immediately after extrusion for a period of time of 30 seconds to 10 minutes.

15. The method as claimed in claim 2, wherein the method is a continuous method.

16. The method as claimed in claim 2, wherein the extruder is a twin screw extruder.

17. The method as claimed in claim 2, wherein the compound resides within the extruder for a residence time of 30 seconds to 15 minutes.

18. The method as claimed in claim 2, wherein the compound is an organic compound with a molecular weight between 100 and 600.
